(19) United States
(12) Patent Application Publication
(10) Pub. No.: US 2024/0301494 A1
(43) Pub. Date: Sep. 12, 2024

(54) IDENTIFICATION AND TREATMENT OF PATIENTS WITH EPILEPSY

(71) Applicant: GW Research Limited, Cambridgeshire (GB)

(72) Inventors: Kevin James CRAIG, Cambridgeshire (GB); Daniel Adam CHECKETTS, Cambridgeshire (GB)

(21) Appl. No.: 18/258,485

(22) PCT Filed: Dec. 17, 2021
(86) PCT No.: PCT/EP2021/086658

§ 371 Date: Jun. 20, 2023

(30) Foreign Application Priority Data
Dec. 21, 2020 (GB) 2020274.3

(51) Int. Cl. C12Q 1/6883

(52) U.S. Cl. CPC C12Q 1/6883; C12Q 2600/106; C12Q 2600/156

(57) ABSTRACT

The present invention relates to the identification of a group of patients with epilepsy who will benefit from treatment with the compound cannabidiol (CBD), and to treatment of that group with CBD. The patients are identified by having variance in specific genes. Also provided is a method for identification of a group of patients with epilepsy at an increased risk of experiencing an adverse event when taking CBD.

### RELATED APPLICATIONS

The present application is related to, and claims the benefit of, GB 2020274.3 filed on 21 Dec. 2020 (21.12.2020), the contents of which are hereby incorporated by reference in their entirety.

### FIELD OF THE INVENTION

The present invention relates to a method for identification of a group of patients with epilepsy who will benefit from treatment with the compound cannabidiol (CBD).

In particular the patients will be identified by genotyping. More specifically the patients will be identified as being having a predisposition to improved or reduced efficacy. In a further embodiment the patient will be identified as being at an increased risk of experiencing an adverse event when taking CBD.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Cannabidiol (CBD), a non-psychoactive derivative from the cannabis plant, has demonstrated anti-convulsant properties in several anecdotal reports, pre-clinical and clinical studies both in animal models and humans. Randomized control trials showed efficacy of the purified pharmaceutical formulation of CBD in patients with Dravet and Lennox-Gastaut syndrome and tuberous sclerosis complex (TSC).

Highly purified cannabidiol (CBD; Epidiolex®) is FDA approved for the treatment of seizures associated with Lennox-Gastaut, Dravet syndromes and Tuberous Sclerosis Complex in patients 1 year and older. However, CBD response remains highly variable, and the mechanisms underlying its therapeutic effects are not fully understood. Although, primarily metabolized by cytochrome P450 (CYP) CYP2C19 and CYP3A4, CBD metabolism is complex. With over 100 metabolites identified, the role of other CYP enzymes, and phase II (glucuronide and sulfate conjugation) pathways in CBD metabolism is being elucidated.

It is recognized that genetic variation in pharmacogenes contributes to variability in drug response, ranging from lack of efficacy, to susceptibility to adverse drug reactions. Identification of genetic predictors of CBD response, both therapeutic and adverse, can help identify patients who could benefit from CBD treatment.

While some studies have examined efficacy of cannabinoids in patients with specific underlying genetic epilepsies, there have not been any studies which have evaluated the influence of genetic variation across drug-related genes on cannabidiol response and tolerability in treatment-resistant epilepsy (TRE).

The present invention has been devised in light of these considerations.

### BRIEF SUMMARY OF THE DISCLOSURE

The applicant has found by way of an open label, expanded-access program where patients were treated with CBD that there was a novel genetic association between genetic variation and efficacy and tolerability of the drug. The study evaluated the association of genetic variants across drug metabolizing enzymes, drug transporters, and other drug-related genes to therapeutic response (≥50% seizure reduction) and tolerability (treatment-associated diarrhea, sedation, and abnormal liver function).

In accordance with a first aspect of the present invention there is provided a method for detecting increased treatment efficacy in a patient with epilepsy comprising the steps of:
a) obtaining a blood sample from the patient with epilepsy;
b) performing a genomics assay on said blood sample;
c) analysing the results of the genomics assay to determine presence of variance in particular genes; and
d) treating said patient with cannabidiol (CBD) if the variance is present.

Preferably the variance is present in one or more of the genes: SULT1A2; CHST11; UGT2B4; ABP1; GSTM5; CYP4Z1; SLC7A7; DPYD; CYP2F1; GSTP1; CYP2D6; ABCG1; ABCC4; ABCC5; CYP1A2; SULT1E1; SLC22A5; SLC7A7; SLC22A3; SLC15A1; FMO2; ADH4; SLCO4A1; SLC28A3; PGAP3; ADH5; COMT; or ADH6.

Preferably the variance is present in a gene associated with Phase I metabolism. In particular the variance is present in one or more of the genes: AOX1; ADH5; ADH6; or DPYD. More preferably the variance is present in the AOX1 gene.

Alternatively, the variance is present in a gene associated with Phase II metabolism. In particular the variance is present in one or more of the genes: SULT1A2; CHST11; or UGT2B4.

In accordance with a second aspect of the present invention there is provided a method for detecting reduced treatment efficacy in a patient with epilepsy comprising the steps of:
a) obtaining a blood sample from the patient with epilepsy;
b) performing a genomics assay on said blood sample;
c) analysing the results of the genomics assay to determine presence of variance in particular genes;
d) not treating said patient with cannabidiol (CBD) if the variance is present.

Preferably the variance is present in one or more of the genes: SULT1A2; CHST11; UGT2B4; ABP1; GSTM5; CYP4Z1; SLC7A7; DPYD; CYP2F1; GSTP1; CYP2D6; ABCG1; ABCC4; ABCC5; CYP1A2; SULT1E1; SLC22A5; SLC7A7; SLC22A3; SLC15A1; FMO2; ADH4; SLCO4A1; SLC28A3; PGAP3; ADH5; COMT; or ADH6.

Preferably the variance is present in a gene associated with Phase I metabolism. More preferably the variance is present in one or more of the genes: CYP1A2; CYP2D6; CYP2F1; or CYP4Z1.

Alternatively, the variance is present in a gene associated with Phase II metabolism. In particular the variance is present in one or more of the genes: SULT1E1; GSTM5; or GSTP1.

In accordance with a third aspect of the present invention there is provided a method for detecting treatment tolerability in a patient with epilepsy comprising the steps of:
a) obtaining a blood sample from the patient with epilepsy;
b) performing a genomics assay on said blood sample;
c) analysing the results of the genomics assay to determine presence of variance in particular genes;
d) treating said patient with cannabidiol (CBD) if variance is present;
e) monitoring said patient for adverse events.

Preferably the variance is present in one or more of the genes: ADH1A; ADH5; ALDH1A1; ALDH3A1; CBR1; CYP2A6; CYP39A1; CYP4F11; CYP8B1; FMO3; FMO6; CHST1; GSTM2; NQO1; SULT1B1; SULT1C2; SULT1E1; UGT2A1; ABCB11; ABCB11; ABCB4; ABCB4; ABCB4; ABCC5; ABCC8; ABCG1; SLC22A1; SLC22A11; SLC22A11; SLC22A2; SLC22A3; SLC22A3; SLC22A5; SLC22A5; SLC28A1; SLCO1B1; SLCO3A1; ABP1; AKAP9; APOA2.

More preferably the variance is present in one or more of the genes: ABCC4; ABCC5; or ABCG1.

Preferably the adverse event is one or more of diarrhoea; sedation or abnormal liver function tests (LFTs).

When the adverse event is diarrhoea the variance is present in one or more of the genes: CYP2A6; CYP39A1; ABCB11; ABCB4; FMO6; SLCO1B1; OATP1B1; SULT1E1; SLC22A3; ABP1.

When the adverse event is sedation the variance is present in one or more of the genes: ADH1A; ADH5; ALDH1A1; ALDH3A1; ABCB11; ABCB4; ABCC5; CYP2C19; ABCB1; COMT; TPMT.

When the adverse event is abnormal liver function tests and the variance is present in one or more of the genes: UGT2A1; SULT1B1; SULT1C2; GSTM2; APOA2; SLC22A2; SLC22A5; SLC22A11.

In a further embodiment the method further comprises the step of decreasing the dose of CBD provided.

In accordance with a fourth aspect of the present invention there is provided a kit for performing genotyping of a patient with epilepsy comprising means for obtaining a blood sample from the patient; means for performing a genomics assay of said blood sample; means to analyse the results of the genomics assay an determine variance in particular genes.

Preferably the kit will identify a variance in a gene that is related to increased efficacy of CBD in a patient. Alternatively, the kit will identify a variance in a gene that is related to decreased efficacy of CBD in a patient. In a further embodiment the kit will identify patients that are related to adverse events in a patient taking CBD.

### BRIEF DESCRIPTION OF THE DRAWINGS

Embodiments of the invention are further described hereinafter with reference to the accompanying drawings, in which:

FIG. 1 shows the genetic variation in drug-related genes associated with CBD response and adverse effects based on permutation p-values (p<0.05).

FIG. 2 shows the receiver operator curves of (a) clinical factors alone (b) genetic factors alone and (c) clinical and genetic factors.

FIG. 3 shows the SNP-SNP interactions among CBD-response related genetic variants included in the genetic model. Black boxes at left represent the response-related variant assessed. Red outlined boxes (SLC28A1 and ADH1C) indicate the interacting variant is also associated with another CBD-related outcome (SLC28A1: sedation, ADH1C: abnormal LFTs). Yellow boxes (ADH6 and CHST1) indicate the gene, but not the specific variant, is associated with a CBD-related outcome (response or adverse effect). The most significant interacting variant identified is presented for each variant (p<0.05)

### DEFINITIONS

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

### DETAILED DESCRIPTION

The present invention relates to the identification of a group of patients with epilepsy in which treatment with cannabidiol (CBD) is associated with improved or reduced efficacy, or with an increased risk of experiencing an adverse event.

### Cannabidiol (CBD)

The CBD used in the methods of the invention may be from any suitable source. Synthetic or botanically-derived CBD may be used. In a preferred embodiment, the botanically-derived CBD is used, such as a botanically-derived purified CBD preparation.

The CBD used in the methods of the invention may have a purity of 95% (w/w) or greater, such as greater than or equal to 98% (w/w). In a preferred embodiment, the CBD used in the methods of the invention comprises less than or equal to 2% (w/w) of other cannabinoids. Other cannabinoids that may be present include THC, CBD-C1, CBDV, and CBD-C4.

### Preparation of Highly Purified CBD Extract

The following describes the production of the highly-purified (>95% w/w) cannabidiol extract which has a known and constant composition.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD. Although the CBD is highly purified because it is produced from a cannabis plant rather than synthetically there is a small number of other cannabinoids which are co-produced and co-extracted with the CBD. Details of these cannabinoids and the quantities in which they are present in the medication are as described in Table A below.

TABLE A
Composition of highly purified CBD extract

| Cannabinoid | Concentration |
|---|---|
| CBD | >95% w/w |
| CBDA | NMT 0.15% w/w |
| CBDV | NMT 1.0% w/W |
| Δ⁹ THC | NMT 0.15% w/w |
| CBD-C4 | NMT 0.5% w/w |

>—greater than
NMT—not more than

### Preparation of Botanically Derived Purified CBD

The following describes the production of the botanically derived purified CBD which comprises greater than or equal to 98% w/w CBD and less than or equal to other cannabinoids was used in the open label, expanded-access program described in Example 1 below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table B and the CBD API is described in Table C.

TABLE B
CBD botanical raw material specification

| Test | Method | Specification |
|---|---|---|
| Identification: | | |
| A | Visual | Complies |
| B | TLC | Corresponds to standard (for |
| | | CBD & CBDA) |
| C | HPLC/UV | Positive for CBDA |
| Assay: | In-house | NLT 90% of assayed |
| CBDA + CBD | (HPLC/UV) | cannabinoids by peak area |
| Loss on Drying | Ph. Eur. | NMT 15% |
| Aflatoxin | UKAS method | NMT 4 ppb |
| Microbial: | | |
| TVC | Ph. Eur. | NMT107 cfu/g |
| Fungi | | NMT105 cfu/g |
| E. coli | | NMT102 cfu/g |
| Foreign Matter: | Ph. Eur. | NMT 2% |
| Residual Herbicides and | Ph. Eur. | Complies |
| Pesticides | | |

TABLE C
Specification of an exemplary botanically derived purified CBD preparation

| Test | Test Method | Limits |
|---|---|---|
| Appearance | Visual | Off-white/pale yellow crystals |
| Identification A | HPLC-UV | Retention time of major peak corresponds to |
| | | certified CBD Reference Standard |
| Identification B | GC-FID/MS | Retention time and mass spectrum of major |
| | | peak corresponds to certified CBD Reference |
| | | Standard |
| Identification C | FT-IR | Conforms to reference spectrum for certified |
| | | CBD Reference Standard |
| Identification D | Melting Point | 65-67° C. |
| Identification E | Specific Optical | Conforms with certified CBD Reference |
| | Rotation | Standard; −110° to −140° (in 95% ethanol) |
| Total Purity | Calculation | ≥98.0% |
| Chromatographic Purity 1 | HPLC-UV | ≥98.0% |
| Chromatographic Purity 2 | GC-FID/MS | ≥98.0% |
| Test | Test Method | Limits |
| CBDA | HPLC-UV | NMT 0.15% w/w |
| CBDV | | NMT 1.0% w/w |
| THC | | NMT 0.1% w/w |
| CBD-C4 | | NMT 0.5% w/w |
| Residual Solvents: | | |
| Alkane | GC | NMT 0.5% w/w |
| Ethanol | | NMT 0.5% w/w |
| Residual Water | Karl Fischer | NMT 1.0% w/w |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:
a) Growing
b) Direct drying
c) Decarboxylation
d) Extraction—using liquid CO₂
e) Winterization using ethanol
f) Filtration
g) Evaporation

High CBD chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ to produce botanical drug substance (BDS), which was then crystalized to produce the test material. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:
a) Crystallization using C₅-C₁₂ straight chain or branched alkane
b) Filtration
c) Vacuum drying

The BDS produced using the methodology above was dispersed in C₅-C₁₂ straight chain or branched alkane. The mixture was manually agitated to break up any lumps and the sealed container then placed in a freezer for approximately 48 hours. The crystals were isolated via vacuum filtration, washed with aliquots of cold C₅-C₁₂ straight chain or branched alkane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in an open label, expanded-access program to investigate the clinical efficacy and safety of purified pharmaceutical cannabidiol formulation (CBD) in the treatment of patients with epilepsy. Patients were enrolled in a genomic study and were genotyped using the Affymetrix Drug Metabolizing Enzymes and Transporters plus array.

The association between variants and CBD response (≥50% seizure reduction) and tolerability was evaluated under dominant and recessive genetic models accounting for known predictors.

### Genetic Variance

The inventors have found that patients with epilepsy, such as TRE, having genetic variance (for example, single-nucleotide polymorphisms; SNPs) in certain genes have altered (such as improved or reduced). outcomes when treated with cannabidiol.

The genetic variants associated with improved or reduced outcomes are present in in one or more of the genes: SULT1A2; CHST11; UGT2B4; ABP1; GSTM5; CYP4Z1; SLC7A7; DPYD; CYP2F1; GSTP1; CYP2D6; ABCG1; ABCC4; ABCC5; CYP1A2; SULT1E1; SLC22A5; SLC7A7; SLC22A3; SLC15A1; FMO2; ADH4; SLCO4A1; SLC28A3; PGAP3; ADH5; COMT; or ADH6.

### Improved Outcomes

The inventors have found that patients with epilepsy, such as TRE, having genetic variance (e.g. SNPs) in certain genes have improved outcomes when treated with cannabidiol.

Accordingly, the invention provides a method for detecting increased treatment efficacy in a patient with epilepsy comprising the steps of:
a) obtaining a blood sample from the patient with epilepsy, such as TRE;
b) performing a genomics assay on said blood sample;
c) analysing the results of the genomics assay to determine presence of variance in particular genes associated with improved outcomes; and optionally
d) treating said patient with cannabidiol (CBD) if the variance is present.

The invention also provides CBD for use in a method of treating a patient with epilepsy, such as TRE, wherein the patient has a genetic variance associated with improved outcomes.

The invention also provides CBD for use in a method of treating a patient with epilepsy, such as TRE, wherein the method comprises:
a) determining whether a blood sample from the patient comprises a genetic variance associated with improved outcomes; and
b) if the blood sample from the patient comprises a genetic variance associated with improved outcomes administering to the patient an effective amount of CBD.

The invention also provides a method of treating epilepsy, such as TRE, comprising administering to a patient in need of treatment a therapeutically effective amount of CBD, wherein the patient has a genetic variance associated with improved outcomes.

The invention also provides use of CBD for the manufacture of a medicament for treating epilepsy, such as TRE, in a patient having a genetic variance associated with improved outcomes.

The generic variance associated with improved outcomes is present in a gene selected from AXO1, SULT1A2, CHST11, UGT2B4, ABP1, SLC7A7, DPYD, ABCG1, ABCC4, SLC7A7, SLCO4A1, SLC28A3, PGAP3, ADH5, COMT, and ADH6.

In one embodiment, the variance associated with improved outcomes is present in a gene associated with Phase I metabolism such as a gene selected from AXO1, DPYD, ADH5, and ADH6. In a preferred embodiment, the variance is present in AXO1.

In one embodiment, the variance associated with improved outcomes is present in a gene associated with Phase II metabolism such as a gene selected from SULT1A2, CHST11, UGT2B4, and COMT. In a preferred embodiment, the variance is present in a gene selected from SULT1A2, CHST11, and UGT2B4.

In one embodiment, the variance associated with improved outcomes is present in a gene associated with transport such as a gene selected from SLC7A7, ABCG1, ABCC4, SLC7A7, SLCO4A1, and SLC28A3.

In one embodiment, the variance associated with improved outcomes is present in a gene selected from ABP1 (AOC1) and PGAP3.

### Reduced Outcomes

The inventors have found that the patients with epilepsy, such as TRE, having genetic variance (for example single-nucleotide polymorphisms; SNPs) in certain genes have reduced outcomes when treated with cannabidiol.

Accordingly, the invention provides a method for detecting reduced treatment efficacy in a patient with epilepsy comprising the steps of:
a) obtaining a blood sample from the patient with epilepsy, such as TRE;
b) performing a genomics assay on said blood sample;
c) analysing the results of the genomics assay to determine presence of variance in particular genes associated with reduced outcomes; and optionally
d) not treating said patient with cannabidiol (CBD) if the variance is present.

The generic variance associated with reduced outcomes is present in a gene selected from GSTM5, CYP4Z1, CYP2F1, GSTP1, CYP2D6, ABCC5, CYP1A2, SULT1E1, SLC22A5, SLC22A3, SLC15A1, FMO2, and ADH4.

In one embodiment, the variance associated with reduced outcomes is present in a gene associated with Phase I metabolism such as a gene selected from CYP4Z1, CYP2F1, CYP2D6, CYP1A2, FMO2, and ADH4. In a preferred embodiment, the variance is present in a gene selected from CYP4Z1, CYP2F1, CYP2D6, and CYP1A2.

In one embodiment, the variance associated with reduced outcomes is present in a gene associated with Phase II metabolism such as a gene selected from GSTM5, GSTP1, and SULT1E1.

In one embodiment, the variance associated with reduced outcomes is present in a gene associated with transport such as a gene selected from ABCC5; SLC22A5; SLC22A3; and SLC15A1.

### Associated Adverse Events

The inventors have found that patients with epilepsy, such as TRE, having genetic variance (e.g. SNPs) in certain genes are at an increased risk of experiencing an adverse event when taking CBD.

Accordingly, the invention provides a method for detecting treatment tolerability in a patient with epilepsy comprising the steps of:
a) obtaining a blood sample from the patient with epilepsy, such as TRE;
b) performing a genomics assay on said blood sample;
c) analysing the results of the genomics assay to determine presence of variance in particular genes associated with adverse events;
d) treating said patient with cannabidiol (CBD) if variance is present; and
e) monitoring said patient for adverse events.

Optionally, the method may further comprise decreasing the dose of CBD provided.

The genetic variance associated with adverse events are present in a gene selected from ADH1A, ADH5, ALDH1A1, ALDH3A1, CBR1, CYP2A6, CYP39A1, CYP4F11, CYP8B1, FMO3, FMO6, CHST1, GSTM2, NQO1, SULT1B1, SULT1C2, SULT1E1, UGT2A1, ABCB11, ABCB4, ABCC5, ABCC8, ABCG1, SLC22A1, SLC22A11, SLC22A2, SLC22A3, SLC22A5, SLC28A1, SLCO1B1, SLCO3A1, ABP1, AKAP9, and APOA2.

In one embodiment, the adverse event is sedation and the associated variance is present in a gene selected from ADH1A, ADH5, ALDH1A1, ALDH3A1, CYP4F11, CYP8B1, FMO3, NQO1, ABCB11, ABCB4, ABCC5, ABCC8, ABCG1, SLC22A5, AKAP9, CYP2C19, ABCB1, COMT, and TPMT. The adverse event may be increased sedation and the associated variance present in a gene selected from ADH1A, ADH5, ALDH1A1, ALDH3A1, CYP4F11, CYP8B1, FMO3, NQO1, ABCB11, ABCB4, ABCC5, ABCC8, ABCG1, SLC22A5, and AKAP9. Alternatively, the adverse event may be decrease sedation and the associated variance present in a gene selected from CYP2C19, ABCB1, COMT, and TPMT.

In one embodiment, the adverse event is abnormal LFTs and the associated variance is present in a gene selected from CBR1, GSTM2, SULT1B1, SULT1C2, UGT2A1, SLC22A11, SLC22A2, SLC22A5, SLCO3A1, and APOA2.

In one embodiment, the adverse event is diarrhoea and the associated variance is present in a gene selected from CYP2A6, CYP39A1, FMO6, CHST1, SULT1E1, ABCB11, ABCB4, SLC22A1, SLC22A11, SLC22A3, SLC28A1, SLCO1B1, and ABP1.

### Drug-Drug Interactions

The inventors also believe that patients with epilepsy, such as TRE, having genetic variance (e.g. SNPs) in certain genes are at an increased risk of experiencing an adverse event when taking CBD in combination with certain other medications.

DPYD is also the primary enzyme responsible for the metabolism of the fluoropyrimidine medications (e.g. fluorouracil, capecitabine).

Accordingly, the present invention provides CBD for use in a method of treating a patient with epilepsy, such as TRE, wherein:
i) the patient has a genetic variance in the gene DPYD; and
ii) the method comprises avoiding concomitant administration of fluoropyrimidine medications, such as fluorouracil and capecitabine.

The invention also provides a method of treating epilepsy, such as TRE, comprising administering to a patient in need of treatment a therapeutically effective amount of CBD, wherein:
i) the patient has a genetic variance in the gene DPYD; and
ii) the method comprises avoiding concomitant administration of fluoropyrimidine medications, such as fluorouracil and capecitabine.

ABP1 (AOC1), encoding diamine oxidase (DAO), generates hydrogen peroxide through degradation of putrescine and histamine. The examples demonstrate that the rs12539 variant was associated with increased response and increased diarrhoea, suggesting that increased histamine levels achieved through impaired histamine degradation may contribute to CBD response and treatment-associated diarrhoea in TRE.

Accordingly, the present invention provides CBD for use in a method of treating a patient with epilepsy, such as TRE, wherein:
i) the patient has a genetic variance in the gene ABP1; and
ii) the method comprises avoiding concomitant administration of antihistamine medications.

The invention also provides a method of treating epilepsy, such as TRE, comprising administering to a patient in need of treatment a therapeutically effective amount of CBD, wherein:
i) the patient has a genetic variance in the gene ABP1; and
ii) the method comprises avoiding concomitant administration of antihistamine medications.

### Other Aspects and Embodiments

Each and every compatible combination of the embodiments described above is explicitly discloses herein, as if each and every combination was individually and explicitly recited.

Carious further aspects and embodiment of the present invention will be apparent to those skilled in the arti in view of the present disclosure.

Where used, “and/or” is to be taken as a specific disclosure of each of the relevant components or features alone as well as a specific disclosure of the combination of the components or features. For example, “A and/or B” is to be taken as specific disclosure of each of i) A, ii) B, and ii) A and B, just as if each were set out individually.

Unless context dictates otherwise, the descriptions and definitions of the features set out above are not limited to any particular aspect or embodiment of the invention and apply equally to all aspects ad embodiments which are described.

### WORKED EXAMPLES

Certain aspects and embodiments of the invention will not be illustrated by way of example and with reference to the figures described above.

### Example 1: The Influence of Variation in Pharmacogenes on Cannabidiol (CBD) Response and Tolerability in Patients with Epilepsy

### Methods

### Study Population:

Patients were enrolled in a compassionate-use open-label CBD (Epidiolex®) study for TRE (May 2018 to March 2019) as part of an Expanded Access Program (EAP). All patients had a diagnosis of TRE confirmed by video/electroencephalography monitoring; an adequate trial/failure of ≥4 AEDs, including ≥1 trial of two concomitant AEDs; and an average of ≥4 seizures per month, averaged over three months.

CBD was initiated at 5 mg/kg/day, administered in divided doses, and titrated up in increments of 5 mg/kg/day approximately every 2 weeks (up to a maximum of 50 mg/kg/day) depending on tolerability and therapeutic response.

### Pharmacogenomic Study:

An additional consent was obtained for the genomic study, and blood samples collected when stable doses of CBD and any concomitant ASDs were reached, with no changes for ≥2 weeks. Baseline data collection included demographics, seizure frequencies/severity, concomitant AEDs, and laboratory data.

All patients (or caregivers) were instructed to keep seizure diaries, which were verified during in-person visits. At each clinic visit, data on concomitant AEDs, CBD maintenance dose (mg/kg/day), seizure frequencies, and adverse events were collected.

### Assessment of CBD Response and Tolerability:

The percent change in seizure frequency was determined at CBD stable maintenance/dose, defined as the time/dose when each patient reached maximal seizure control, and calculated as ([seizure frequency per 28 days]−[seizure frequency at baseline])/[seizure frequency at baseline]×100.

Based on percent change, response was categorized: no change/increase, >0 to 25%, >25% to <50%, >50% to <75%, and >75% reduction, with CBD response defined as ≥50% reduction from baseline.

Adverse effects were classified according to the Medical Dictionary for Regulatory Activities (MedDRA, V17.1), and those that differed by response status (diarrhoea, sedation, and abnormal liver function tests (LFTs)) were evaluated in the genetic analyses.

### Genotyping and Quality Control:

CLIA-certified genotyping on the Affymetrix Drug Metabolizing Enzymes and Transporters (DMET) plus array was performed through the Coriell Institute for Medical Research (Camden, NJ) in two batches, with nine samples re-genotyped due to low call rates. The DMET plus array interrogates 1,936 markers (1,931 variants and 5 copy number regions) across ˜230 drug-related genes. Affymetrix DMET Console software® (version 1.3) was used to generate genotype profiles. Of the 1,931 variants, 1,328 were removed due to location on the X-chromosome or a minor allele frequency <0.05.

Hardy-Weinberg equilibrium was evaluated, and variants p<0.0001 were further evaluated by race. Variants in linkage disequilibrium (LD) were removed (n=212; r2=0.5), leaving 396 variants in the analyses.

### Statistical Analysis:

Statistical analyses were performed using PLINK version 1.9 and SAS version 9.4 (Cary, NC). After stratification by response status, differences in patient characteristics were assessed using analysis of variance models for continuous variables and χ2 tests for categorical variables. Due to the number and combinations of concomitant ASDs, their limited influence on response, and low interaction potential, only ASDs expected to influence pharmacokinetic properties or have additive toxicity (e.g. valproate and hepatotoxicity) with CBD and used by >10% of patients were evaluated. This included clobazam, valproate, zonisamide, topiramate, and rufinamide.

Relationships between variants and CBD response/tolerability were tested under dominant (AA vs Ab+bb) and recessive (AA+Ab vs bb) models using logistic regression in PLINK. Treatment group (adult vs paediatric), race, and sex were included as covariates in all analyses. The relationship between clinical predictors and outcomes was evaluated using linear (CBD dose) and logistic regression, and variables p<0.2 were included as covariates in the corresponding genetic analysis.

Odds ratios (OR) and 95% confidence intervals (CI) were calculated for CBD response and adverse effects. A permutation approach was used to account for multiple testing (number of permutations: 1,000), since other methods (e.g. Bonferroni correction) assume independence between markers and likely be overly conservative.

A multivariable CBD response model incorporated treatment group, sex, race, CBD dose, and genetic variants p<0.05 using backward selection, with the final model retaining statistically significant predictors. Receiver operator curves (ROC) were generated and the area under the curve (AUC) was evaluated to compare clinical, genetic, and combined clinical+genetic models. Epistasis was evaluated in PLINK to identify potential interactions among variants included in the final response model.

### Results

### Study Population:

Of the 169 patients in the open-label study 113 participated in the genomic study. One patient was excluded due to sex discordance, resulting in 112 patients (54-5% paediatric; 50.0% female).

Therapeutic response (≥50% reduction) was achieved for 56.3% (63 of 112) patients with a mean CBD dose of 26.62±14.24 mg/kg/day. There were no significant differences in clinical characteristics by response status (Table 1).

On average, patients had tried and failed eight ASDs and were on three concomitant AEDs. Responders were more likely to experience diarrhoea (p=0.01) and abnormal LFTs (p=0.05); whereas non-responders were more likely to experience sedation (p=0.05). The majority of patients (80.4%) experienced some degree of weight loss independent of therapeutic response.

TABLE 1
Demographic and clinical characteristics between CBD responders and non-responders

| | | | | | Non- | | |
|---|---|---|---|---|---|---|---|
| | Overall | | Responders | | Responders | | |
| | (N = 112) | | (N = 63) | | (N = 49) | | |
| Demographics | N (%) | | N (%) | | N (%) | | p-value |
| Treatment Group | | | | | | | |
| Adult | 51 | (45.5%) | 30 | (47.6%) | 21 | (42.9%) | 0.62 |
| Paediatric | 61 | (54.5%) | 33 | (52.4%) | 28 | (57.1%) | |
| Gender | | | | | | | |
| Female | 56 | (50.0%) | 34 | (54.0%) | 22 | (44.9%) | 0.34 |
| Male | 56 | (50.0%) | 29 | (46.0%) | 27 | (55.1%) | |
| Self-reported race | | | | | | | |
| African American | 15 | (13.4%) | 10 | (15.9%) | 5 | (10.2%) | 0.65 |
| White | 94 | (83.9%) | 52 | (82.5%) | 42 | (85.7%) | |
| Other | 3 | (0.03%) | 1 | (0.02%) | 2 | (0.04%) | |
| Baseline | | | | | | | |
| Characteristics | Mean ± SD | | Mean ± SD | | Mean ± SD | | |
| Age | 20.96 ± 16.11 | | 19.76 ± 14.72 | | 22.50 ± 17.77 | | 0.39 |
| Age at epilepsy onset | 5.50 ± 9.82 | | 5.37 ± 7.56 | | 5.67 ± 12.22 | | 0.88 |
| Weight (kg) | 55.02 ± 28.88 | | 55.70 ± 29.71 | | 54.13 ± 28.07 | | 0.77 |
| Bilirubin (mg/dL) | 0.34 ± 0.16 | | 0.32 ± 0.13 | | 0.37 ± 0.19 | | 0.13 |
| ALT | 24.10 ± 12.06 | | 24.03 ± 11.19 | | 24.19 ± 13.17 | | 0.95 |
| AST | 27.88 ± 14.53 | | 27.41 ± 11.71 | | 28.47 ± 17.54 | | 0.72 |
| Seizure frequencies | 136.26 ± 442.52 | | 132.62 ± 407.24 | | 140.94 ± 488.45 | | 0.92 |
| Median (IQR) | 23.05 | (6.36, 65.4) | 29 | (10, 68.7) | 13.7 | (5.2, 56.7) | 0.18 |
| ASDs tried/failed | 8.32 ± 3.48 | | 8.25 ± 3.35 | | 8.41 ± 3.67 | | 0.82 |
| Number of concomitant | 2.64 ± 0.99 | | 2.67 ± 1.03 | | 2.61 ± 0.95 | | 0.77 |
| AEDs | | | | | | | |
| Concomitant AEDs N (%) | | | | | | | |
| clobazam | 40 | (35.7%) | 21 | (33.3%) | 19 | (38.8%) | 0.55 |
| valproate | 21 | (18.8%) | 13 | (20.6%) | 8 | (16.3%) | 0.56 |
| zonisamide | 20 | (17.9%) | 13 | (20.6%) | 7 | (14.3%) | 0.38 |
| topiramate | 18 | (16.1%) | 10 | (15.9%) | 8 | (16.3%) | 0.95 |
| rufinamide | 12 | (10.7%) | 6 | (9.5%) | 6 | (12.2%) | 0.64 |
| Adverse Events* | N (%) | | N (%) | | N (%) | | |
| Diarrhoea | 66 | (58.9%) | 44 | (69.8%) | 22 | (44.9%) | 0.01 |
| Sedation | 39 | (34.8%) | 17 | (27.0%) | 22 | (44.9%) | 0.05 |
| Nausea/vomiting | 14 | (12.5%) | 9 | (14.3%) | 5 | (10.2%) | 0.52 |
| Abnormal liver function | 12 | (10.7%) | 10 | (15.9%) | 2 | (4.1%) | 0.05 |
| tests | | | | | | | |
| Depressed mood | 12 | (10.7%) | 5 | (7.9%) | 7 | (14.3%) | 0.28 |
| Decreased appetite | 12 | (10.7%) | 9 | (14.3%) | 3 | (6.1%) | 0.17 |
| Weight Change | | | | | | | |
| No Change or increase | 22 | (19.6%) | 14 | (22.2%) | 8 | (16.3%) | 0.67 |
| <10% Weight loss | 58 | (51.8%) | 32 | (50.8%) | 26 | (53.1%) | |
| 10-20% Weight loss | 23 | (20.5%) | 11 | (17.5%) | 12 | (24.5%) | |
| >20% Weight loss | 9 | (8.0%) | 6 | (9.5%) | 3 | (6.1%) | |
| Treatment-Related | | | | | | | |
| Measures | Mean ± SD | | Mean ± SD | | Mean ± SD | | p-value |
| CBD dose (mg/kg/day) | 23.81 ± 14.21 | | 26.62 ± 14.24 | | 20.20 ± 13.46 | | 0.02 |
| Maintenance seizure | 58.54 ± 222.27 | | 19.65 ± 46.09 | | 108.52 ± 327.05 | | 0.07 |
| frequency | | | | | | | |
| Median (IQR) | 7.76 | (2.58, 32.2) | 4.50 | (1.28, 13.6) | 14.73 | (5.67, 51) | <0.001 |
| Percent change seizure | −42.08 ± 58.76 | | −79.20 ± 16.58 | | 5.65 ± 59.13 | | <0.001 |
| frequency | | | | | | | |
| Median (IQR) | −53.9 | (−85·3, −17.1) | −82 | (−94, −63·3) | −14.3 | (−32−4, 2·7) | <0.001 |

AED: anti-epilepsy drug
*Other adverse effects <10% in the cohort included rash (n = 11), upper respiratory infection (n = 8), hospital admission (n = 7), hyponatremia (n = 6), and abnormal CBC (n = 5)

### Genetic Predictors of Response:

Variants associated with response after accounting for LD, treatment group, sex, race, and CBD dose are presented in Table 2. In the single-marker analysis, the most significant association was identified for the phase I aldehyde oxidase, AOX1 gene at SNP rs6729738, which is recessive. Variance in this gene was associated with a 6-fold increased likelihood of response (OR 6.39, 95% CI 2.13-19.17; p=0.001).

Conversely X, genetic variation in the CYP enzymes, including CYP1A2 rs762551, CYP2D6 rs28371725, CYP2F1 rs305968 (recessive) and CYP4Z1 rs7512729, was associated with a lower likelihood of response. For CYP1A2 rs762551 (*1F), lack of two variant A alleles, conferring higher enzyme inducibility, was associated with decreased odds of response (p=0.03).

Phase I dehydrogenases including alcohol (ADH5 rs2602836) and dihydropyrimidine (DPYD rs1801265) were also associated with an increased response.

Among phase II pathways, SULT1A2 rs1059491 (recessive), was associated with a 17-fold increased likelihood of response (p=0.004); whereas SULT1E1 rs3775770 was associated with lower odds of response (p=0.03). Variation in the carbohydrate sulfotransferase, CHST11 rs903247, and the glucuronosyltransferase, UGT2B4 rs1966151, was associated with 4-fold increased odds of response (p-values <0.01). Conversely, variation in glutathione-S-transferases, including GSTM5 rs2479390 and GSTP1 rs1695 (recessive), were associated with 72% and 87% decreased likelihood of response, respectively (p≤0.01).

Drug transporters identified included ATP binding cassette (ABC) and solute carrier (SLC) family transporters. SLC transporters included the Y+L amino acid transporter (SLC7A7), a carnitine transporter (SLC22A5), the extraneuronal monoamine transporter (SLC22A3), and a nucleoside transporter (SLC28A3). Response-associated variants in the ABC transporters ABCC4, ABCC5, and ABCG1, were also associated with a CBD-related adverse effect. Additionally, variation in ABP1 (AOC1), involved in histamine degradation, was also implicated in response.

TABLE 2
Genetic variants associated with CBD response (≥50% seizure reduction) under dominant and recessive
genetic models adjusted for treatment group (adult vs paediatric), sex, race, and CBD dose (mg/kg/day)

| | | | Genetic | | R | NR | Odds Ratio | | EMP |
|---|---|---|---|---|---|---|---|---|---|
| Gene | SNP | CHR | model | Genotype | (n) | (n) | [95% CI] | p-value | p-value |
| AOX1 | rs6729738 | 2 | recessive | cc | 24 | 6 | 6.39 [2.13-19.17] | 0.0009 | 0.001 |
| | | | | Tc + TT | 39 | 43 | | | |
| SULT1A2 | rs1059491 | 1 | recessive | gg | 13 | 1 | 17.72 [2.07-150.3] | 0.008 | 0.004 |
| | | | | Tg + TT | 50 | 48 | | | |
| CHST11 | rs903247 | 12 | dominant | Ct + cc | 39 | 17 | 3.92 [1.69-9.14] | 0.002 | 0.004 |
| | | | | TT | 24 | 32 | | | |
| UGT2B4 | rs1966151 | 4 | dominant | Ct + cc | 32 | 11 | 4.15 [1.67-10.34] | 0.002 | 0.002 |
| | | | | TT | 31 | 38 | | | |
| ABP1 | rs12539 | 7 | dominant | Ct + tt | 30 | 12 | 3.65 [1.52-8.75] | 0.004 | 0.003 |
| | | | | CC | 31 | 37 | | | |
| GSTM5 | rs2479390 | 1 | dominant | Ag + gg | 14 | 23 | 0.28 [0.12-0.69] | 0.005 | 0.008 |
| | | | | AA | 49 | 26 | | | |
| CYP4Z1 | rs7512729 | 1 | dominant | Ac + cc | 35 | 37 | 0.29 [0.11-0.74] | 0.01 | 0.01 |
| | | | | AA | 25 | 12 | | | |
| SLC7A7 | rs1061040 | 14 | dominant | Ga + aa | 24 | 10 | 3.55 [1.31-9.64] | 0.01 | 0.02 |
| | | | | GG | 39 | 39 | | | |
| DPYD | rs1801265 | 1 | dominant | Ct + cc | 35 | 15 | 2.96 [1.3-6.74] | 0.01 | 0.02 |
| | | | | TT | 28 | 34 | | | |
| CYP2F1 | rs305968 | 19 | recessive | aa | 3 | 11 | 0.18 [0.05-0.73] | 0.02 | 0.01 |
| | | | | Ga + GG | 60 | 38 | | | |
| GSTP1 | rs1695 | 11 | recessive | gg | 2 | 8 | 0.13 [0.02-0.75] | 0.02 | 0.01 |
| | | | | Ag + AA | 61 | 41 | | | |
| CYP2D6 | rs28371725 | 22 | dominant | Ga + aa | 5 | 11 | 0.24 [0.07-0.8] | 0.02 | 0.02 |
| | | | | GG | 57 | 37 | | | |
| ABCG1 | rs914189 | 21 | dominant | Cg + gg | 26 | 11 | 2.81 [1.17-6.77] | 0.02 | 0.02 |
| | | | | CC | 36 | 38 | | | |
| ABCC4 | rs2274406 | 13 | dominant | Ga + aa | 47 | 27 | 2.66 [1.13-6.27] | 0.03 | 0.02 |
| | | | | GG | 16 | 22 | | | |
| ABCC5 | rs3749442 | 3 | dominant | Ct + tt | 16 | 22 | 0.39 [0.17-0.91] | 0.03 | 0.04 |
| | | | | CC | 47 | 27 | | | |
| CYP1A2 | rs762551 | 15 | dominant | Ac + cc | 26 | 31 | 0.41 [0.19-0.92] | 0.03 | 0.03 |
| | | | | AA | 37 | 18 | | | |
| SULT1E1 | rs3775770 | 4 | dominant | Ga + aa | 23 | 26 | 0.41 [0.18-0.92] | 0.03 | 0.03 |
| | | | | GG | 40 | 23 | | | |
| SLC22A5 | rs1045020 | 5 | dominant | Ct | 7 | 14 | 0.32 [0.11-0.90] | 0.03 | 0.03 |
| | | | | CC | 56 | 35 | | | |
| SLC7A7 | rs1805061 | 14 | dominant | Tc + cc | 23 | 9 | 2.85 [1.10-7.41] | 0.03 | 0.02 |
| | | | | TT | 39 | 36 | | | |
| SLC22A3 | rs668871 | 6 | dominant | Ct + tt | 45 | 34 | 0.39 [0.16-0.93] | 0.03 | 0.04 |
| | | | | CC | 18 | 15 | | | |
| SLC15A1 | rs1339067 | 13 | recessive | tt | 1 | 6 | 0.09 [0.01-0.80] | 0.03 | 0.003 |
| | | | | Ct + CC | 62 | 43 | | | |
| FMO2 | rs7515157 | 1 | recessive | tt | 4 | 7 | 0.20 [0-05-0.90] | 0.04 | 0.01 |
| | | | | Ct + CC | 59 | 42 | | | |
| ADH4 | rs3762894 | 4 | recessive | gg | 1 | 6 | 0.10 [0.01-0.90] | 0.04 | 0.01 |
| | | | | Ag + AA | 62 | 43 | | | |
| SLCO4A1 | rs2236553 | 20 | recessive | cc | 14 | 4 | 3.71 [1.07-12.81] | 0.04 | 0.04 |
| | | | | TT + Tc | 49 | 45 | | | |
| SLC28A3 | rs7867504 | 9 | recessive | gg | 10 | 2 | 5.50 [1.07-28.29] | 0.04 | 0.02 |
| | | | | Ag + AA | 52 | 43 | | | |
| PGAP3 | rs2952151 | 17 | recessive | aa | 12 | 3 | 4.29 [1.05-17.49] | 0.04 | 0.04 |
| | | | | GG + Ga | 49 | 45 | | | |
| ADH5 | rs2602836 | 4 | recessive | tt | 20 | 7 | 2.87 [1.04-7.96] | 0.04 | 0.05 |
| | | | | CC + Ct | 43 | 42 | | | |
| COMT | rs4680 | 22 | recessive | gg | 19 | 8 | 2.67 [1.00-7.13] | 0.05 | 0.06 |
| | | | | AA + Ag | 41 | 40 | | | |
| ADH6 | rs10002894 | 4 | dominant | Tc + cc | 28 | 12 | 2.35 [1.00-5.49] | 0.05 | 0.06 |
| | | | | TT | 35 | 37 | | | |

CHR: chromosome
R: CBD responder (≥50% reduction in seizure frequency)
NR: CBD non-responder
EMP p-value: empirical p-value obtained from 1,000 permutations

### Genetic Predictors of CBD-Associated Adverse Effects:

Variants associated with an increased likelihood of developing CBD-associated adverse effects are presented in Table 3, and those associated with increased and decreased likelihood of each adverse effect (including variants in LD).

### CBD-Associated Diarrhoea.

After accounting for treatment group, sex, race, baseline weight, and clobazam, genetic variation in CYP2A6 rs28399433, involved in coumarin, nicotine, and caffeine metabolism, was associated with a 10-fold increased likelihood of diarrhoea. Additionally, CYP39A1 rs7761731, involved in neural cholesterol clearance through conversion to bile acids, and FMO6 rs2272797, a pseudogene, were associated with an approximate 9-fold and 5-fold increased likelihood of diarrhoea, respectively. Additionally, the ABC transporters ABCB11 rs3770603, encoding the bile salt export pump, and ABCB4 rs4148808, responsible for the transport of phospholipids into bile, shared the same association with diarrhoea as FMO6 (OR 5·48 95% CI 1.52-19.75; p=0.01). Increased odds of diarrhoea were also observed for patients with SLCO1B1 rs11045819, encoding the OATP1B1 transporter, and involved in endogenous bile acid transport (OR 3.55, 95% CI 1.11-11.36; p=0.03). Among variants related to response, SULT1E1 rs3775770, SLC22A3 rs668871, and ABP1 rs12539 were also associated with increased odds of CBD-related diarrhoea.

### CBD-Associated Sedation.

After accounting for treatment group, sex, race, CBD dose, clobazam and rufinamide co-therapy, patients harbouring variants in phase I alcohol (ADH1A rs6811453 and ADH5 rs7687322) and aldehyde (ALDH1A1 rs13959 and ALDH3A1 rs2072330) dehydrogenases had increased likelihood of sedation. Similar to diarrhoea, variation in the ABCB11 (rs4148768) and ABCB4 (rs2097937 and rs4148807) transporters was associated with increased sedation. ABCC5 rs3749442 associated with decreased response was associated with increased sedation. Multiple genes recognized as important pharmacogenes in the pharmacogenomics knowledgebase were related to decreased sedation, including CYP2C19 rs3758581 (p=0.004), ABCB1 rs1045642 (p=0.003), COMT rs4680 recessive wild-type G alleles (p=0.03), and TPMT rs2842934 (p=0.03).

CBD-associated abnormal LFTs.

After adjustment for treatment group, sex, race, and baseline weight, variants related to the development of abnormal LFTs were largely concentrated in phase II metabolic pathways including glucuronide (UGT2A1 rs4148304), sulphate (SULT1B1 rs1604741 and SULT1C2 rs17036104), and glutathione (GSTM2 rs592792) conjugation. Additionally, recessive variants at rs5085 in apolipoprotein A2, APOA2, associated with hypercholesterolemia, were associated with a 6-fold increased likelihood of abnormal LFTs (OR 6.45, 95% CI 1.24-33.59; p=0.03). Recessive variants in the SLC22A family genes, SLC22A2 rs316003, SLC22A5 rs274548, and SLC22A11 rs1783811, involved in renal drug secretion, were associated with approximate 11-fold, 15-fold, and 6-fold increased odds of abnormal LFTs, respectively.

UGT2A1, involved in bile detoxification, was associated with diarrhoea, increased LFTs and sedation. The rs4148304 missense variant was associated with increased odds of developing abnormal LFTs and decreased odds of sedation, whereas the rs11249454 intron variant was associated with decreased odds of diarrhoea.

TABLE 3
Genetic variants associated with increased likelihood of experiencing CBD-related adverse effects

| | | | | | Genetic | Adverse | OR | | EMP |
|---|---|---|---|---|---|---|---|---|---|
| | Gene | SNP | CHR | EA | model | effect | [95% CI] | p-value | p-value |
| Phase I | ADH1A | rs6811453 | 4 | T | recessive | sedation | 8.66 [1.90-39.49] | 0.005 | 0.002 |
| Metabolism | ADH5 | rs7687322 | 4 | T | dominant | sedation | 5.14 [1.05-25.26] | 0.04 | 0.03 |
| | ALDH1A1 | rs13959 | 9 | T | dominant | sedation | 4.73 [1.27-17.67] | 0.02 | 0.03 |
| | ALDH3A1 | rs2072330 | 17 | T | dominant | sedation | 3.27 [1.16-9.23] | 0.025 | 0.02 |
| | CBR1 | rs998383 | 21 | G | dominant | abnormal | 12.31 [1.50-101.4] | 0.02 | 0.02 |
| | | | | | | LFTs | | | |
| | CYP2A6 | rs28399433 | 19 | G | dominant | diarrhoea | 9.52 [2.15-42.06] | 0.003 | 0.001 |
| | CYP39A1 | rs7761731 | 6 | A | recessive | diarrhoea | 9.25 [1.45-59.14] | 0.02 | 0.01 |
| | CYP4F11 | rs1060463 | 19 | G | dominant | sedation | 3.22 [1.08-9.60] | 0.04 | 0.04 |
| | CYP8B1 | rs6774801 | 3 | A | recessive | sedation | 4.45 [1.08-18.41] | 0.04 | 0.03 |
| | FMO3 | rs2066534 | 1 | G | dominant | sedation | 5.33 [1.92-14.80] | 0.001 | 0.004 |
| | FMO6 | rs2272797 | 1 | G | dominant | diarrhoea | 5.48 [1.52-19.75] | 0.009 | 0.01 |
| Phase II | CHST1 | rs9787901 | 11 | T | dominant | diarrhoea | 6.12 [1.56-24.04] | 0.009 | 0.005 |
| Metabolism | GSTM2 | rs592792 | 1 | T | dominant | abnormal | 4.45 [1.19-16.64] | 0.03 | 0.02 |
| | | | | | | LFTs | | | |
| | NQO1 | rs689453 | 16 | A | dominant | sedation | 4.65 [1.28-16.91] | 0.02 | 0.01 |
| | SULT1B1 | rs1604741 | 4 | C | recessive | abnormal | 6.30 [1.54-25.75] | 0.01 | 0.004 |
| | | | | | | LFTs | | | |
| | SULT1C2 | rs17036104 | 2 | G | dominant | abnormal | 4.65 [1.23-17.65] | 0.02 | 0.02 |
| | | | | | | LFTs | | | |
| | SULT1E1 | rs3775770 | 4 | A | recessive | diarrhoea | 10.78 [1.02-114.20] | 0.05 | 0.02 |
| | UGT2A1 | rs4148304 | 4 | A | Dominant | abnormal | 4.75 [1.23-18.33] | 0.02 | 0.01 |
| | | | | | | LFTs | | | |
| Transport | ABCB11 | rs3770603 | 2 | A | dominant | diarrhoea | 5.48 [1.52-19.75] | 0.009 | 0.01 |
| | ABCB11 | rs4148768 | 2 | T | dominant | sedation | 4.38 [1.13-16.92] | 0.03 | 0.025 |
| | ABCB4 | rs4148808 | 7 | G | dominant | diarrhoea | 5.48 [1.52-19.75] | 0.009 | 0.01 |
| | ABCB4 | rs2097937 | 7 | C | recessive | sedation | 11.07 [1.51-81.09] | 0.02 | 0.01 |
| | ABCB4 | rs4148807 | 7 | T | dominant | sedation | 4.07 [1.19-13.91] | 0.025 | 0.02 |
| | ABCC5 | rs3749442 | 3 | T | dominant | sedation | 3.30 [1.20-9.10] | 0.02 | 0.02 |
| | ABCC8 | rs757110 | 11 | G | recessive | sedation | 6.07 [1.27-29.06] | 0.02 | 0.02 |
| | ABCG1 | rs1541290 | 21 | G | recessive | sedation | 3.99 [1.30-12.26] | 0.02 | 0.02 |
| | SLC22A1 | rs628031 | 6 | A | recessive | diarrhoea | 6.12 [1.24-30.25] | 0.03 | 0.03 |
| | SLC22A11 | rs2078267 | 11 | T | recessive | diarrhoea | 4.84 [1.34-17.49] | 0.02 | 0.03 |
| | SLC22A11 | rs1783811 | 11 | A | recessive | abnormal | 5.85 [1.13-30.37] | 0.04 | 0.04 |
| | | | | | | LFTs | | | |
| | SLC22A2 | rs316003 | 6 | G | recessive | abnormal | 16.30 [1.69-157.00] | 0.02 | 0.02 |
| | | | | | | LFTs | | | |
| | SLC22A3 | rs668871 | 6 | T | dominant | diarrhoea | 3.11 [1.16-8.38] | 0.025 | 0.03 |
| | SLC22A3 | rs2292334 | 6 | A | recessive | diarrhoea | 5.78 [1.23-27.07] | 0.03 | 0.03 |
| | SLC22A5 | rs274548 | 5 | T | recessive | abnormal | 12.74 [1.04-156.1] | 0.05 | 0.02 |
| | | | | | | LFTs | | | |
| | SLC22A5 | rs274548 | 5 | T | dominant | sedation | 3.25 [1.18-8.94] | 0.02 | 0.03 |
| | SLC28A1 | rs2242046 | 15 | A | recessive | diarrhoea | 8.47 [1.93-37.20] | 0.005 | 0.007 |
| | SLCO1B1 | rs11045819 | 12 | A | dominant | diarrhoea | 3.55 [1.11-11.36] | 0.03 | 0.03 |
| | SLCO3A1 | rs2283458 | 15 | A | recessive | abnormal | 5.17 [1.04-25.56] | 0.04 | 0.04 |
| | | | | | | LFTs | | | |
| Other | ABP1 | rs12539 | 7 | T | dominant | diarrhoea | 2.98 [1.11-8.02] | 0.03 | 0.04 |
| | AKAP9 | rs7785971 | 7 | A | recessive | sedation | 4.09 [1.08-15.46] | 0.04 | 0.02 |
| | APOA2 | rs5085 | 1 | C | recessive | abnormal | 5.72 [1.15-28.43] | 0.03 | 0.03 |
| | | | | | | LFTs | | | |

### Shared Genetic Predictors of CBD Response and Adverse Effects:

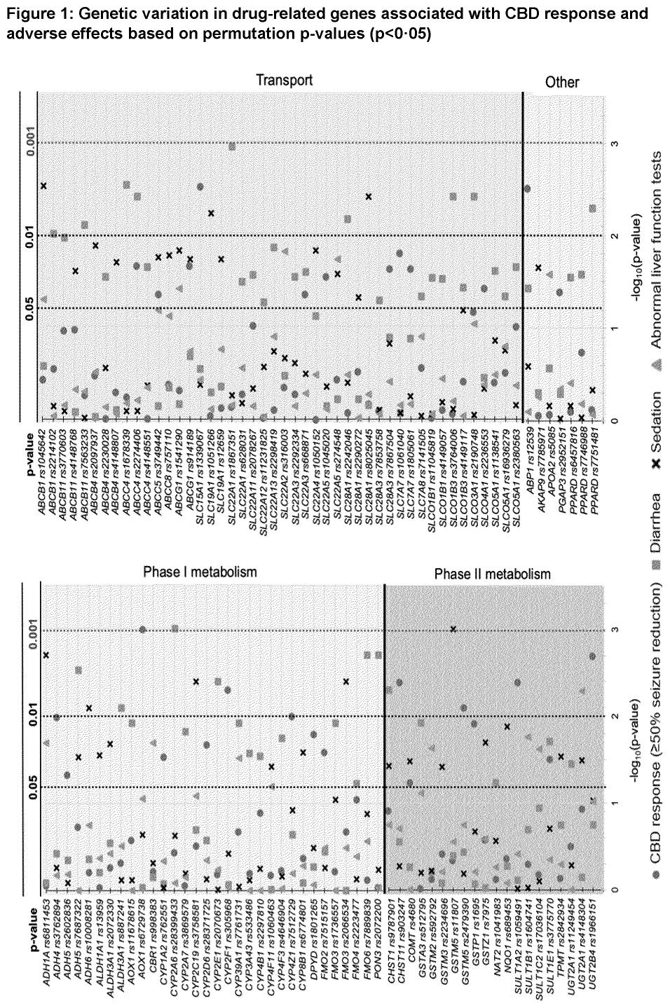

FIG. 1 presents pharmacogenes associated with CBD response and adverse outcomes). For phase I and II metabolic pathways, different variants were responsible for the observed effects and included ADH5 and AOX1 (phase I metabolism), and SULT1E1 and GSTM5 (phase II metabolism). For drug transporters, the same variants were implicated in both response and tolerability and included ABCC4 rs2274406, ABCC5 rs3749442, ABCG1 rs914189, and SLC22A3 rs668871. Similarly, ABP1 (AOC1) rs12539, was associated with increased response and diarrhoea.

### Multivariable CBD Response Model:

Across all assessments, treatment group, sex, and race did not remain significant and were not included in the final multivariable model (Table 4). Significant variants were primarily located in phase II metabolic pathways and included a sulfotransferase (CHST11 rs903247), glucuronosyltransferase (UGT2B4 rs1966151), and glutathione-S transferases (GSTM5 rs2479390 and GSTP1 rs1695).

While no CYP enzymes remained significant, the phase I dehydrogenase, DPYD rs1801265, and flavin-containing monooxygenase, FMO2 rs7515157, were included in the final model. SLC7A7 rs1061040, encoding the Y+L amino acid transporter, was the only transporter significant after multivariable analysis. Additionally, ABP1 (AOC1) remained significant in the final model (p=0.007)

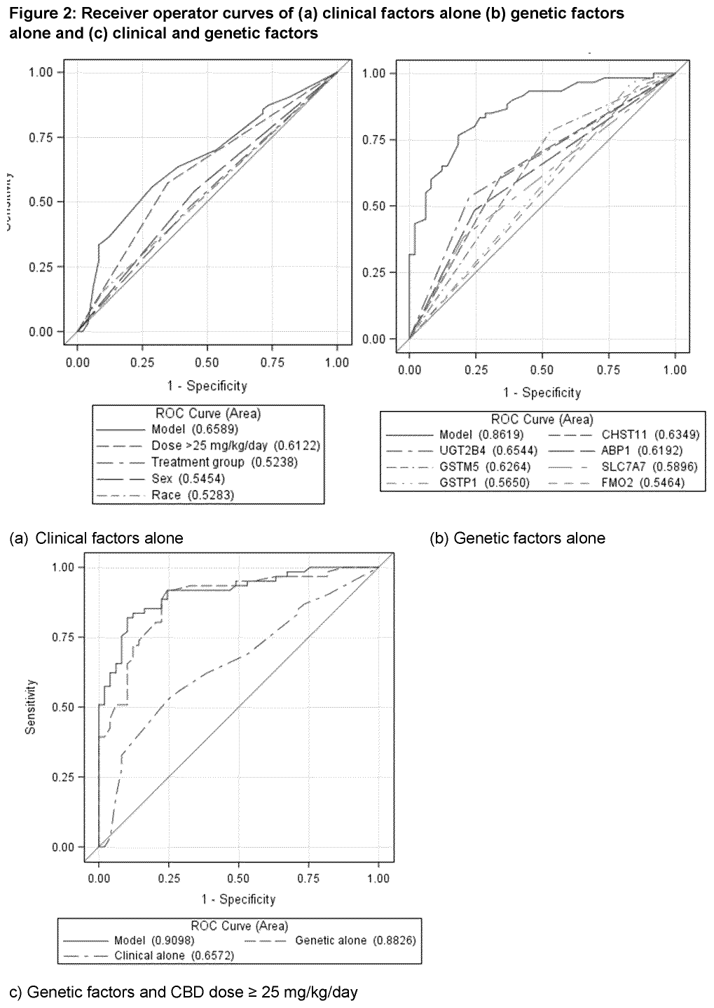

Models incorporating clinical factors (FIG. 2a), genetic factors (FIG. 2b), and clinical+genetic factors (FIG. 2c) were constructed using a CBD dose threshold of ≥25 mg/kg/day. Compared to clinical factors alone (AUC 0.659, 95% CI 0.56-0.76), the genetic model resulted in an AUC of 0.883 (95% CI 0.82-0.95; p=0.003), and the combined model resulted in an AUC of 0.9098 (95% CI 0.86-0.96; p<0.0001). There was no statistically significant difference between the genetic model alone and the combined clinical+genetic model (p=0·14).

### SNP×SNP Interactions:

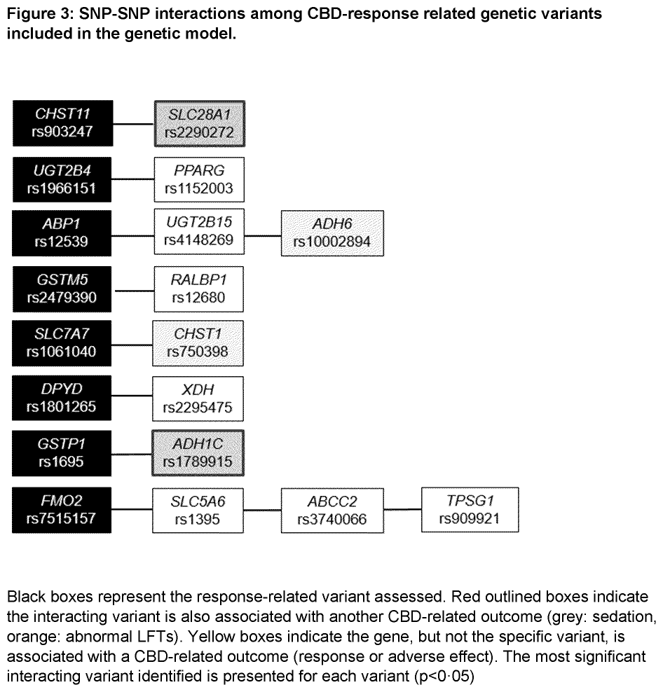

Epistasis was identified for all variants included in the final model (FIG. 3). Among all variants in the study, the most significant interaction was observed between UGT2B4 rs1966151 and the nuclear receptor, PPARγ rs1152003 (p=1.46×10⁻⁵). Additionally, the response-related variants CHST11 rs903247 and GSTP1 rs1695 had interactions with SLC28A1 rs2290272, a pyrimidine transporter associated with decreased sedation, and ADH1C rs1789915, an alcohol dehydrogenase associated with decreased abnormal LFTs, respectively. SLC7A7 rs1061040 had an interaction with CHST1 rs750398, of which another variant (rs9787901) was associated with diarrhoea and sedation.

TABLE 4
Multivariable model of CBD response in treatment-
resistant epilepsy and role of implicated genes

| Gene | Role | Biologic function | OR | 95% CI | p-value |
|---|---|---|---|---|---|
| CBD dose (mg/kg/day) | | NA | 1.07 | [1.03- | 0.002 |
| | | | | 1.12] | |
| DPYD | Phase I | Involved in the degradation of | 4.50 | [1.40- | 0.01 |
| rs1801265 | metabolism | pyrimidine bases. Rate limiting | | 14.52] | |
| | | step in uracil catabolism | | | |
| FMO2 | Phase I | Involved in innate immune | 0.06 | [0.01- | 0.01 |
| rs7515157 | metabolism | response and modulation of | | 0.52] | |
| (recessive) | | oxidative stress. Increases | | | |
| | | release of reactive oxygen | | | |
| | | species through hydrogen | | | |
| | | peroxide generation. | | | |
| CHST11 | Phase II | Catalyses the transfer of | 5.03 | [1.52- | 0.008 |
| rs903247 | metabolism | sulphate to N- | | 16.61] | |
| | | acetylgalactosamine of | | | |
| | | chondroitin | | | |
| UGT2B4 | Phase II | Involved in bile acid | 5.02 | [1.60- | 0.006 |
| rs1966151 | metabolism | conjugation. Active on | | 15.80] | |
| | | oestrogens and xenobiotics. | | | |
| GSTM5 | Phase II | Detoxifies electrophilic | 0.15 | [0.04- | 0.004 |
| rs2479390 | metabolism | compounds (carcinogens, | | 0.55] | |
| | | drugs, environmental toxins, | | | |
| | | and products of oxidative | | | |
| | | stress) | | | |
| GSTP1 | Phase II | Involved in glutathione | 0.03 | [0.002- | 0.005 |
| rs1695 | metabolism | conjugation of prostaglandins | | 0.34] | |
| (recessive) | | A2 and J2. Prevents | | | |
| | | neurodegeneration through | | | |
| | | negative regulation of CDK5 | | | |
| SLC7A7 | Transport | Cationic amino acid transporter. | 5.04 | [1.38- | 0.01 |
| rs1061040 | | Involved in glutamine influx | | 18.47] | |
| ABP1 | Other | Catalyses the degradation of | 5.67 | [1.61- | 0.007 |
| (AOC1) | | putrescine, histamine, and | | 20.03] | |
| rs 12539 | | other substances involved in | | | |
| | | allergic and immune response | | | |

### CONCLUSIONS

The main reasons for withdrawal from CBD expanded access studies are lack of efficacy and adverse events. The data presented in the above example add valuable knowledge and support the incorporation of genetic information to help identify patients with TRE who could benefit from CBD therapy.

These data surprisingly demonstrate that variability in CBD response is primarily due to genetic factors, with clinical factors having little influence on therapeutic response among patients with TRE.

As has previously been reported these data support the complex CBD metabolism, with genetic variation across phase I and II pharmacogenes implicated in response and tolerability. Furthermore, variation in genetics which results in influencing drug transport is likely to contribute substantially to variability in response.

Importantly, these data demonstrate that pharmacogenes play a vital role in the regulation of fundamental biologic processes, independent of their role in drug metabolism/transport, and alteration in these pathways may contribute to underlying pathologic processes. As such there are potential interactions between CBD and commonly used medications (e.g. statins, acetaminophen, antihistamines) that may require caution with concomitant use as discussed in more detail below.

Nucleotide catabolism: the rate limiting step in uracil catabolism leading to the formation of β-alanine, a structural analogue of the inhibitory neurotransmitter gamma-aminobutyric acid. Genetic variation in DPYD can result in dihydropyrimidine dehydrogenase deficiency, which can manifest as neurologic complications including seizures. DPYD is also the primary enzyme responsible for the metabolism of the fluoropyrimidine medications (fluorouracil, capecitabine). While DPYD-mediated response may be due to the underlying biologic process, and not CBD metabolism, patients should be monitored for toxicity if fluoropyrimidines and CBD are co-administered. DPYD was observed to have an epistatic interaction with xanthine dehydrogenase (XDH), involved in purine metabolism. AOX1, the most significant gene associated with response in the single-marker analysis, is a paralog of XDH, and has been implicated in the development of amyotrophic lateral sclerosis. AOX1 is involved in the metabolism of tryptophan, nicotinamide and retinaldehyde, and can catalyze the formation of hydrogen peroxide and superoxide. CBD has been shown to decrease reactive oxygen species, and this may serve as an important mechanism underlying response in TRE.

Chondroitin and keratan sulphate proteoglycans: Variants in the carbohydrate sulfotransferases (CHSTs) were associated with treatment response (CHST11), and diarrhoea and sedation (CHST1). Additionally, CHST1 was identified through an epistatic interaction with SLC7A7. CHSTs sulphate proteoglycans, and CHST11 and CHST1 sulphate chondroitin and keratan sulphate, respectively. Chondroitin and keratan sulphate proteoglycans are important components of the brain extracellular matrix, and are involved in the formation of perineuronal nets, which provide neuroprotection and play a role in neuronal development and plasticity. Remodelling of chondroitin sulphate proteoglycans has been implicated in neurologic conditions including epilepsy, and Alzheimer's disease. Given that CHST genes were identified across CBD-related outcomes, carbohydrate-dependent mechanisms likely contribute to CBD response and tolerability.

Bile acid conjugation and cholesterol transport: Altered bile acid signalling pathways have been implicated in the development of various neurodegenerative diseases. Variation in UGT2 family genes, involved in bile acid conjugation, was associated with therapeutic response (UGT2B4) and all adverse effects (UGT2A1). In addition to bile acid conjugation, UGT2B4 is active on catechol-oestrogens, or endogenous estrogenic metabolites. Catechol-oestrogens modulate calcium influx and insulin secretion through activation of the transient receptor potential (TRP) A1 channel. TRPs are involved in neuronal signalling, and TRP channels (vanilloid, ankyrin, and melastatin), are modulated by cannabinoids. Additionally, SULT1E1, associated with decreased response and increased diarrhoea, is responsible for oestrogen inactivation via sulphate conjugation, suggesting a role for steroid hormones. UGT2B4 was also involved in a significant epistatic interaction with PPARγ, highly expressed in adipose tissue and involved in glucose metabolism and lipid storage. CBD has been shown to activate PPARγ, a mechanism associated with its anti-inflammatory and neuroprotective effects. Given that approximately 80% of participants experienced weight loss, PPARγ activation may contribute to these effects and deserves further evaluation.

Variation in bile acid pathways was strongly associated with diarrhoea, including CYP39A1, responsible for converting neural cholesterol into bile acids, and the bile acid-associated transporters ABCB11, ABCB4, and SLCO1B1. Additionally, recessive variants at rs5085 in APOA2, associated with increases in cholesterol levels, was associated with abnormal LFTs. Given that APOA2 contributes to hypercholesterolemia, and SLCO1B1 is involved in statin transport, a potential CBD-statin interaction exists with respect to endogenous bile acid transport. Further evaluation of any potential additive hepatotoxicity is needed and closer monitoring of LFTs in patients on CBD-statin co-therapy. Based on these results, bile acid and cholesterol-associated pathways may play a role in CBD response and diarrhoea.

Glutathione conjugation: Variants in the phase II glutathione-S-transferases (GSH) were associated with decreased likelihood of response (GSTM5 and GSTP1), abnormal LFTs (GSTM2), diarrhoea (GSTA3), and sedation (GSTM3, GSTM5, and GSTZ1). Glutathione acts as an antioxidant and free radical scavenger, and decreased glutathione levels have been implicated in neurodegenerative conditions including epilepsy, Alzheimer's disease, and multiple sclerosis. CBD has been shown to increase GSH activity, thereby decreasing oxidative stress. While it is not clear if glutathione depletion contributes to CBD resistance, or if variation in GSTM5 and GSTP1 impairs CBD-mediated increases in GSH activity, glutathione conjugation appears to contribute substantially to CBD response and tolerability. Given that glutathione conjugation is a saturable pathway, interactions may exist with other drugs that utilize this pathway, such as acetaminophen. As such the role of agents conjugated with glutathione on CBD efficacy and/or tolerability should be monitored closely.

Histamine degradation: ABP1 (AOC1), encoding diamine oxidase (DAO), generates hydrogen peroxide through degradation of putrescine and histamine. Histamine functions as a neurotransmitter and is also involved in gastric acid secretion. Decreased histamine levels have been observed in patients with Alzheimer's disease, and are associated with the development of seizures. Given that the rs12539 variant was associated with increased response and increased diarrhoea, increased histamine levels achieved through impaired histamine degradation, may contribute to CBD response and treatment-associated diarrhoea in TRE. Therefore, the use of antihistamines should be carefully considered when CBD is given for TRE, as histamine receptor blockade may contribute to reduced efficacy.

In summary these data indicate that genetic variation in pharmacogenes is strongly associated with CBD response and the development of adverse effects in TRE. Such data can help identify TRE patients who could benefit from CBD therapy and those patients who are more likely to have an adverse event.

1. A method for detecting increased treatment efficacy in a patient with epilepsy comprising the steps of:
a) obtaining a blood sample from the patient with epilepsy;
b) performing a genomics assay on said blood sample;
c) analysing the results of the genomics assay to determine presence of variance in particular genes associated with improved outcomes; and
d) treating said patient with cannabidiol (CBD) if the variance is present,
wherein the variance associated with improved outcomes is present in a gene selected from AXO1, SULT1A2, CHST11, UGT2B4, ABP1, SLC7A7, DPYD, ABCG1, ABCC4, SLC7A7, SLCO4A1, SLC28A3, PGAP3, ADH5, COMT, and ADH6.

2. The method of claim 1, wherein the variance associated with improved outcomes is present in a gene associated with Phase I metabolism such as a gene selected from AXO1, DPYD, ADH5, and ADH6.

3. The method of claim 2, wherein the variance is present in the AOX1 gene.

4. The method of claim 1, wherein the variance associated with improved outcomes is present in a gene associated with Phase II metabolism such as a gene selected from SULT1A2, CHST11, UGT2B4, and COMT.

5. The method of claim 4, wherein the variance is present in a gene selected from SULT1A2, CHST11, and UGT2B4.

6. The method of claim 1, wherein the variance associated with improved outcomes is present in a gene associated with transport such as a gene selected from SLC7A7, ABCG1, ABCC4, SLC7A7, SLCO4A1, and SLC28A3.

7. The method of claim 1, wherein the variance associated with improved outcomes is present in a gene selected from ABP1 (AOC1) and PGAP3.

8. CBD for use in a method of treating a patient with epilepsy, wherein the patient has a genetic variance in a gene selected from AXO1, SULT1A2, CHST11, UGT2B4, ABP1, SLC7A7, DPYD, ABCG1, ABCC4, SLC7A7, SLCO4A1, SLC28A3, PGAP3, ADH5, COMT, and ADH6.

9. The CBD for use of claim 8, wherein the variance is present in a gene associated with Phase I metabolism such as a gene selected from AXO1, DPYD, ADH5, and ADH6.

10. The CBD for use of claim 9, wherein the variance is present in the AOX1 gene.

11. The CBD for use of claim 8, wherein the variance is present in a gene associated with Phase II metabolism such as a gene selected from SULT1A2, CHST11, UGT2B4, and COMT.

12. The CBD for use of claim 11, wherein the variance is present in a gene selected from SULT1A2, CHST11, and UGT2B4.

13. The CBD for use of claim 8, wherein the variance associated with improved outcomes is present in a gene associated with transport such as a gene selected from SLC7A7, ABCG1, ABCC4, SLC7A7, SLCO4A1, and SLC28A3.

14. The CBD for use of claim 8, wherein the variance associated with improved outcomes is present in a gene selected from ABP1 (AOC1) and PGAP3.

15. A method for detecting reduced treatment efficacy in a patient with epilepsy comprising the steps of:
a) obtaining a blood sample from the patient with epilepsy;
b) performing a genomics assay on said blood sample;
c) analysing the results of the genomics assay to determine presence of variance in particular genes associated with reduced outcomes; and
d) not treating said patient with cannabidiol (CBD) if the variance is present,
wherein the variance associated with reduced outcomes is present in a gene selected from GSTM5, CYP4Z1, CYP2F1, GSTP1, CYP2D6, ABCC5, CYP1A2, SULT1E1, SLC22A5, SLC22A3, SLC15A1, FMO2, and ADH4.

16. The method of claim 15, wherein the variance associated with reduced outcomes is present in a gene associated with Phase I metabolism such as a gene selected from CYP4Z1, CYP2F1, CYP2D6, CYP1A2, FMO2, and ADH4.

17. The method of claim 15, wherein the variance associated with reduced outcomes is present in a gene associated with Phase II metabolism such as a gene selected from GSTM5, GSTP1, and SULT1E1.

18. The method of claim 15, wherein the variance associated with reduced outcomes is present in a gene associated with transport such as a gene selected from ABCC5; SLC22A5; SLC22A3; and SLC15A1.

19. A method for detecting treatment tolerability in a patient with epilepsy comprising the steps of:
a) obtaining a blood sample from the patient with epilepsy;
b) performing a genomics assay on said blood sample;
c) analysing the results of the genomics assay to determine presence of variance in particular genes associated with adverse events;
d) treating said patient with cannabidiol (CBD) if variance is present; and
e) monitoring said patient for adverse events,
wherein the genetic variance associated with adverse events are present in a gene selected from ADH1A, ADH5, ALDH1A1, ALDH3A1, CBR1, CYP2A6, CYP39A1, CYP4F11, CYP8B1, FMO3, FMO6, CHST1, GSTM2, NQO1, SULT1B1, SULT1C2, SULT1E1, UGT2A1, ABCB11, ABCB4, ABCC5, ABCC8, ABCG1, SLC22A1, SLC22A11, SLC22A2, SLC22A3, SLC22A5, SLC28A1, SLCO1B1, SLCO3A1, ABP1, AKAP9, and APOA2.

20. The method of claim 19, wherein the adverse event is diarrhoea and the variance is present in a gene selected from CYP2A6, CYP39A1, FMO6, CHST1, SULT1E1, ABCB11, ABCB4, SLC22A1, SLC22A11, SLC22A3, SLC28A1, SLCO1B1, and ABP1.

21. The method of claim 19, wherein the adverse event is sedation and the variance is present in a gene selected from ADH1A, ADH5, ALDH1A1, ALDH3A1, CYP4F11, CYP8B1, FMO3, NQO1, ABCB11, ABCB4, ABCC5, ABCC8, ABCG1, SLC22A5, AKAP9, CYP2C19,

22. The method of claim 19, wherein the adverse event is abnormal liver function tests and the variance is present in a gene selected from CBR1, GSTM2, SULT1B1, SULT1C2, UGT2A1, SLC22A11, SLC22A2, SLC22A5, SLCO3A1, and APOA2.

23. The method of any of claims 19 to 22, further comprising the step of decreasing the dose of CBD provided.

24. A kit for performing genotyping of a patient comprising means for obtaining a blood sample from a patient with epilepsy; means for performing a genomics assay of said blood sample; means to analyse the results of the genomics assay and determine variance in particular genes.

25. The kit of claim 24, characterised in that the variance in a gene is related to increased efficacy of CBD in a patient.

26. The kit of claim 24, characterised in that the variance in a gene is related to decreased efficacy of CBD in a patient.

27. The kit of claim 24, characterised in that the variance in a gene is related to adverse events in a patient using CBD.
